(19) United States
(12) Patent Application Publication
(10) Pub. No.: US 2023/0191067 A1
(43) Pub. Date: Jun. 22, 2023

(54) EXPIRATORY FILTER WITH EMBEDDED DETECTORS

(71) Applicant: Covidien LP, Mansfield, MA (US)

(72) Inventors: Daniele Resca, San Felice Sul Panaro (IT); Daniel Patrick Wade, Longmont, CO (US); Maurizio Listo, Modena (IT); Matthew J. Phillips, Carlsbad, CA (US); Michael Tyler Wedeking, Denver, CO (US); Lucio Ialonardi, San Felice Sul Panaro (IT); Alessandro Di Mauro, Sesto San Giovanni (IT)

(73) Assignee: Covidien LP, Mansfield, MA (US)

(21) Appl. No.: 18/065,061

(22) Filed: Dec. 13, 2022

Related U.S. Application Data
(60) Provisional application No. 63/292,573, filed on Dec. 22, 2021.

(51) Int. Cl. A61M 16/10; A61M 16/00

(52) U.S. Cl. CPC A61M 16/105; A61M 16/0003; A61M 16/024; A61M 2016/0042

(57) ABSTRACT

Systems and methods for collecting breathing gas properties via a medical ventilatory filter and wirelessly transmitting the data to another device. For example, the filter includes a first housing enclosing filtration media for filtering breathing gases flowing through the filter, the first housing defining a first port and a second port exposed to the breathing gases; and a sensor assembly. The sensor assembly includes a first sensor coupled to the first port, the first sensor configured to capture measurement data for a first gas property of breathing gases flowing through the filter; a second sensor coupled to the second port, the second sensor configured to capture measurement data for a first gas property of the breathing gases flowing through the filter; and a second housing. The second housing includes a processor and communication circuitry operative to wirelessly communicate the sensor data to a computing device located remotely from the filter.

### CROSS-REFERENCE TO RELATED APPLICATIONS

This application claims the benefit of U.S. Provisional Application No. 63/292,573 filed Dec. 22, 2021, entitled “Expiratory Filter with Embedded Detectors,” which is incorporated herein by reference in its entirety.

### INTRODUCTION

Medical ventilator systems have long been used to provide ventilatory and supplemental oxygen support to patients. These ventilators typically comprise a source of pressurized oxygen which is fluidly connected to the patient through a conduit or tubing.

### SUMMARY

This disclosure describes systems and methods for providing real-time gas property data from a breathing gas filter of a ventilation system. In an aspect, the technology relates to a medical ventilatory filter. The filter includes a first housing enclosing filtration media for filtering breathing gases flowing through the filter, the first housing defining a first port and a second port exposed to the breathing gases; and a sensor assembly. The sensor assembly includes a first sensor coupled to the first port, the first sensor configured to capture measurement data for a first gas property of breathing gases flowing through the filter; a second sensor coupled to the second port, the second sensor configured to capture measurement data for a first gas property of the breathing gases flowing through the filter; and a second housing. The second housing includes a processor, enclosed within the second housing, operative to process sensor data received from the first sensor and the second sensor; communication circuitry, in communication with the processor, operative to wirelessly communicate the sensor data to a computing device located remotely from the filter; a first physical input interface physically coupling the first sensor to the second housing and electrically coupling the first sensor and the processor; and a second physical input interface physically coupling the second sensor to the second housing and electrically coupling the second sensor and the processor.

In an example, the first sensor is removable from at least one of the first housing or the second housing. In another example, the first sensor is permanently embedded into the first housing. In a further example, the second housing is removably attached to at least one of the first sensor or the second sensor. In yet another example, the first physical input interface allows for removing the first sensor from the second housing. In still another example, the first sensor is positioned upstream from the filtration media and the second sensor is positioned downstream from the filtration media.

In another example, the first sensor is one of a temperature sensor or a carbon dioxide sensor. In a further example, the first sensor is a carbon dioxide sensor. In still another example, a first end of the filter is configured to connect to first portion of a breathing circuit of a ventilation system and a second end of the filter is configured to connect to a second portion of the breathing circuit. In still yet another example, the filter is configured to connect to a breathing circuit of a ventilation system between a patient interface and a wye-fitting. In another example, the first sensor is configured to measure gas properties associated with exhaled breathing gases.

In another aspect, the present technology relates to a method for providing real-time gas property data of gases flowing through a medical ventilation system. The method includes initiating, by a data acquisition device having a first housing enclosing a controller and wireless communication circuitry, wireless communication session with a remote application; measuring, by a sensor physically coupled to the first housing and second housing of a filter enclosing filter media for filtering breathing gases flowing through the filter, a gas property of the breathing gases; receiving, by the data acquisition device from the sensor, the measurement of the gas property of breathing gases; and wirelessly transmitting, by the wireless communication circuitry, the received gas property measurement to the remote application.

In an example, the sensor is removably connected to the first housing of the data acquisition device via an input interface of the first housing. In another example, receiving, from the sensor, the measurement of the gas property comprises receiving a temperature measurement of the exhaled breathing gases. In yet another example, receiving, from the sensor, the measurement of the gas property comprises receiving a carbon dioxide level measurement of the ventilated patient's exhaled breathing gases. In still another example, the filter is positioned between a patient interface and a wye-fitting of the ventilation system.

In another aspect, the technology relates to a ventilation system that includes a pneumatic system having an inhalation port and an exhalation port; an inhalation limb connected to the inhalation port; an exhalation limb connected to the exhalation port; a wye-fitting connected to the inhalation limb and the exhalation limb; a patient interface; and a first filter positioned between a patient and the wye-fitting. The first filter includes a first filter housing enclosing filtration media for filtering breathing gases flowing through the filter, the first filter housing defining a first port exposed to the breathing gases flowing through the first filter housing; a first sensor coupled to the first port, the first sensor configured to capture measurement data for a first gas property of breathing gases flowing through the first filter; and a first data-acquisition housing. The first data acquisition includes a first processor, enclosed within the first data-acquisition housing, operative to process sensor data received from the first sensor; and first communication circuitry, in communication with the first processor, operative to wirelessly communicate the sensor data from the first sensor to a computing device located remotely from the ventilation system; and a first physical input interface physically coupling the first sensor to the first data-acquisition housing and electrically coupling the first sensor and the first processor.

In an example, the system further includes a second filter positioned on the exhalation limb between the wye-fitting and the pneumatic system. The second filter further includes a second filter housing enclosing filtration media for filtering breathing gases flowing through the filter, the second filter housing defining a second port exposed to the breathing gases flowing through the second filter housing; a second sensor coupled to the second port, the second sensor configured to capture measurement data for a second gas property of breathing gases flowing through the second filter; and a first data-acquisition housing including: a first processor, enclosed within the first data-acquisition housing, operative to process sensor data received from the first sensor; first communication circuitry, in communication with the first processor, operative to wirelessly communicate the sensor data from the first sensor to a computing device located remotely from the ventilation system; and a first physical input interface physically coupling the first sensor to the first data-acquisition housing and electrically coupling the first sensor and the first processor.

In an example, the first sensor is removable from the first filter housing. In another example, the first data-acquisition housing is removable from the first sensor.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the present disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims appended hereto.

FIG. 1 is schematic diagram illustrating an example ventilation system in accordance with aspects of the disclosure.

FIG. 2 is another schematic diagram illustrating an example ventilation system in accordance with aspects of the disclosure.

FIG. 3A depicts a side of an example filter comprising sensor assembly in accordance with aspects of the disclosure.

FIG. 3B is an exploded view of the example filter of FIG. 3A in accordance with aspects of the disclosure.

FIG. 4 is a flow chart of an example method for collecting real-time gas property data via a ventilator filter and wirelessly transmitting the data to another device in accordance with aspects of the disclosure.

### DETAILED DESCRIPTION

Medical ventilators are used to provide breathing gases to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gases having a desired concentration of oxygen are supplied to the patient at desired pressures and rates. Ventilators capable of operating independently of external sources of pressurized air are also available. Increasing communicative connectivity of medical devices to computer networks can provide healthcare clinicians with systems that can provide beneficial and life-saving features. For example, wireless communication technology implemented into various components of a ventilator may enable a transmission of real-time health parameter data to clinicians and data analytics devices.

For instance, while operating a ventilator, it may be desirable to obtain real-time gas property data such as temperature and/or capnography (e.g., carbon dioxide level) measurements. In some examples, that real-time gas property data, such as the capnography data, may be indicative of patient conditions or patient health. The gas property data may also be indicative of ventilator operations. The measured gas property data may be provided to medical personnel who may rely on such measurements to make decisions related to care of the patient.

It may be further desirable to obtain certain measurements, such as exhaled breathing gas characteristic measurements, from a location closer to the patient's mouth or nose, which may provide more accurate measurements than measurements taken farther away from the patient. Current monitoring devices, such as capnography monitors, however, result in significant increases in cost and complexity of the ventilation system.

The present technology, among other things, reduces the need for external monitoring devices, such as capnography devices, and still provides real-time gas property data, including measurements made near the patient's mouth or nose. For instance, the present technology integrates sensors into a filter that may be positioned near the patient's mouth or nose to obtain exhaled breathing gas characteristic measurements. The exhaled breathing gas characteristic measurements may then be wirelessly transmitted to another device to provide real-time information when monitoring the patient and/or for use in adjusting ventilator settings. In some examples, one or more components of the filter, such as filter material and sensor components, may be disposable, while other components, such as data transmission components may be removable from the filter and reused.

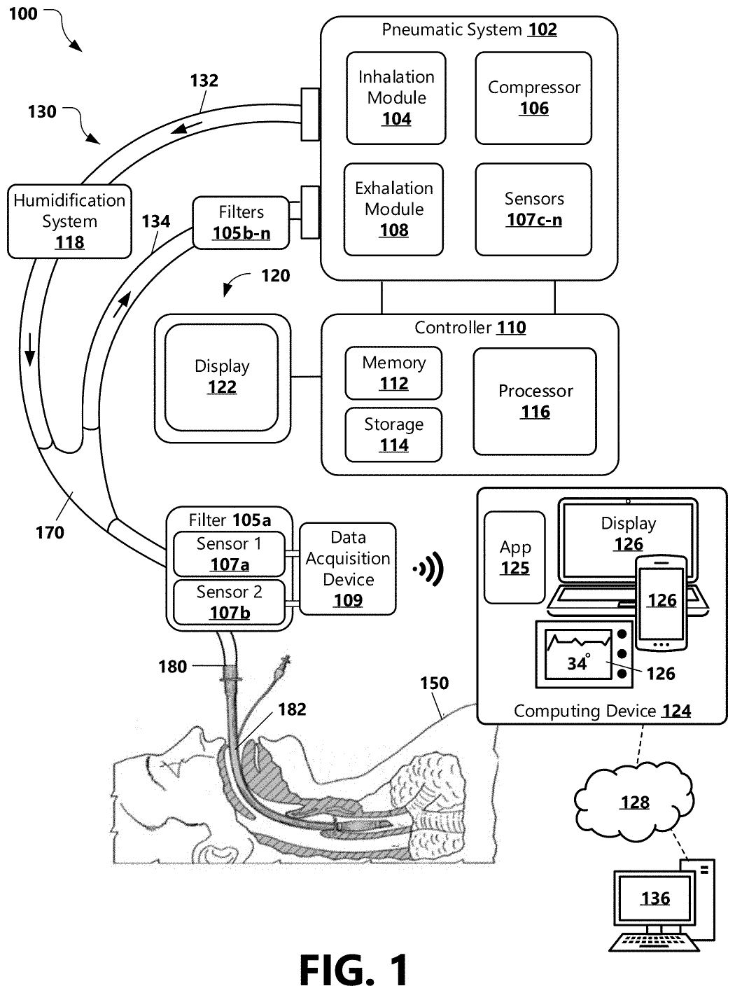

FIG. 1 is a diagram illustrating an example of a medical ventilation system 100 connected to a human patient 150. The medical ventilation system 100 may provide positive pressure ventilation to the patient 150. The medical ventilation system 100 includes a pneumatic system 102 (also referred to as a pressure generating system) for circulating breathing gases to and from the patient 150 via the ventilation tubing system 130, which couples the patient 150 to the pneumatic system via a patient interface 180, such as an invasive (e.g., endotracheal tube 182, as shown) or a non-invasive (e.g., nasal mask, nasal cannula) patient interface.

The ventilation tubing system 130 may be a two-limb (shown) or a one-limb circuit for carrying gases to and from the patient 150. In a two-limb example, a fitting, typically referred to as a “wye-fitting” 170 or “wye,” may be provided to couple the patient interface 180 to an inhalation limb 132 and an exhalation limb 134 of the ventilation tubing system 130. In the example shown in FIG. 1, the patient interface 180 is an endotracheal tube 182 coupled to the ventilation tubing with a connector. The depicted patient interface 180 (e.g., endotracheal tube 182 and connector) are used for invasive ventilation of a patient 150. Other patient interfaces may be implemented, such as non-invasive interfaces, including a nasal cannula, a mask, a nasal mask, etc.

The pneumatic system 102 may have a variety of configurations. In the present example, the pneumatic system 102 includes an exhalation module 108 coupled with the exhalation limb 134 and an inhalation module 104 coupled with the inhalation limb 132. A compressor 106 or other source(s) of pressurized gases (e.g., air, oxygen, and/or helium) is coupled with the inhalation module 104 to provide a gas source for ventilatory support via the inhalation limb 132. The pneumatic system 102 may include a variety of other components, including mixing modules, valves, sensors 107a-n (generally 107), tubing, accumulators, filters 105a-n (generally, 105), etc., which may be internal or external to the ventilation system 100 (and, in some examples, may be communicatively coupled, or capable of communicating, with the ventilation system 100).

A controller 110 is operatively coupled with the pneumatic system 102, signal measurement and acquisition systems, and an operator interface 120 that may enable an operator to interact with the ventilation system 100 (e.g., change ventilation settings, select operational modes, view monitored parameters, etc.). The controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device. In some examples, the display 122 included in the operator interface 120 may provide various input screens for receiving clinician input and various display screens for presenting useful information to the clinician. In one aspect, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilation system 100 may be provided, for instance by a wheel, keyboard, mouse, voice recognition, or other suitable interactive device. Thus, the operator interface 120 may accept commands and input through the display 122. The display 122 may also provide useful information in the form of various ventilatory data regarding the physical condition of the patient 150 (e.g., patient data). In some examples, the useful information may be derived by the ventilation system 100, based on data collected by the processor(s) 116, and the useful information may be displayed to the clinician in the form of graphs, wave representations, pie graphs, text, or other suitable forms of graphic display. For example, patient data may be displayed on the GUI and/or display 122. In one aspect, the patient data may display one or more of a flow rate, a relative humidity of the breathing gases, a temperature of the breathing gases, a selected breath type, a humidifier on or a humidifier off status, etc. Additionally or alternatively, patient data may be communicated to a remote monitoring system coupled via any suitable means to the ventilation system 100.

The example ventilation system 100 illustrated in FIG. 1 shows a first filter 105a positioned between the patient 150 and the wye-fitting 170 and one or more other filters 105b-n positioned along the exhalation limb 134 of the ventilation tubing system 130. In other examples the one or more other filters 105b-n may be placed in any suitable location, e.g., within the pneumatic system 102, ventilation tubing system 130, affixed or embedded in or near the wye-fitting 170 and/or patient interface 180, coupled to the inhalation and/or exhalation modules 104,108, etc. In some examples, at least the first filter 105a of the one or more filters 105a-n can include or connect to a data acquisition device 109 that may operate to collect various parameters/measurement data from sensors 107 that the data acquisition device 109 is communicatively connected to and transmit the measurement data wirelessly to a computing device 124. The data acquisition device 109 may be external to the filter 105, for example, and may attach to the filter 105 to communicate with one or more sensors 107 included in the filter 105. In some examples, the data acquisition device 109 may operate to transmit sensor measurement data to the computing device 124 using BLUETOOTH, BLUETOOTH Low Energy (BLE), ZIGBEE, ANT, Z-WAVE, or another close proximity wireless communication protocol.

The computing device 124, for example, can include a mobile computing device, a desktop computing device, a server computing device, a home-care ventilator device, a central monitoring station, a wearable computing device, or another type of computing device that may operate to receive the sensor measurement data from the data acquisition device 109. In some examples, the computing device 124 may further operate to run an application 125 that may derive or generate useful information based on the received sensor measurement data.

As mentioned above, the ventilation system 100 may include one or more sensors 107, which may be internal or external to the pneumatic system 102. The sensors 107 may be placed in any suitable location, e.g., within the ventilatory circuitry or other devices attached to the ventilation system 100. For instance, the sensors 107 may be located in the pneumatic system 102, ventilation tubing system 130, affixed or embedded in or near the wye-fitting 170 and/or patient interface 180, integrated in or attached to one or more filters 105, coupled to the inhalation module 104 and/or exhalation module 108, on the patient 150, etc. In some examples, one or more sensors 107 may detect changes in gas properties that are indicative of patient parameters utilized in patient monitoring and/or for controlling breath delivery (e.g., triggering breaths). Example sensors 107 may include a humidity sensor, a temperature sensor, a combined temperature/humidity sensor, a carbon dioxide (CO₂) sensor, and/or inspiratory flow sensor. Indeed, any sensory device useful for monitoring changes in measurable parameters during ventilatory treatment may be employed in accordance with aspects described herein.

According to one example implementation, a first sensor 107a may include a temperature sensor and a second sensor 107b may include a carbon dioxide (CO₂) sensor. As illustrated in FIG. 1, the first sensor 107a and the second sensor 107b may be included in a first filter 105a positioned between the patient 150 and the wye-fitting 170. The first filter 105a may be one of one or a plurality of filters 105a-n that may be connected to components of the ventilation system 100. In some aspects, the first filter 105a may be placed between the patient 150 and the wye-fitting 170 such that the first sensor 107a and the second sensor 107b may be near the patient 150 to more accurately identify exhaled breathing gas characteristics, such as the temperature and carbon dioxide level of the airways or lungs of the patient 150. For example, exhaled breathing gas characteristic measurements taken closer to the patient 150 may be more accurate than measurements that may be taken farther away from the patient 150, for example, due to localized temperature and/or carbon dioxide level changes. Thus, the exhaled breathing gas characteristic measurements may be more accurate when monitoring the patient 150 and/or for use in ventilation system 100 settings.

According to one example, measurement data (e.g., parameters) collected by the data acquisition device 109 from the first sensor 107a and the second sensor 107b and transmitted to the computing device 124 may include characteristics of the exhaled breathing gases. According to another example, measurement data (e.g., parameters) collected by the data acquisition device 109 from the first sensor 107a and the second sensor 107b and transmitted to the computing device 124 may include characteristics of the delivered breathing gases. The application 125 may operate to display received measurement data and/or derive use useful information based on the breathing gas measurements. In an example, the useful information derived by the application 125 may be used for evaluating the patient's condition when ventilating the patient 150, for ventilator settings, and/or for correlative assessments.

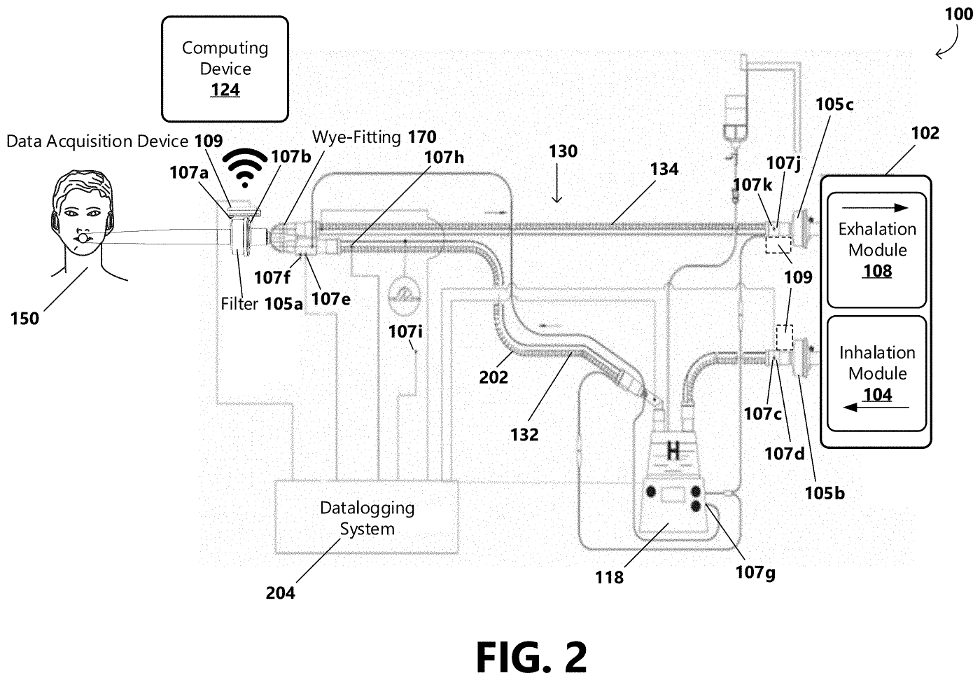

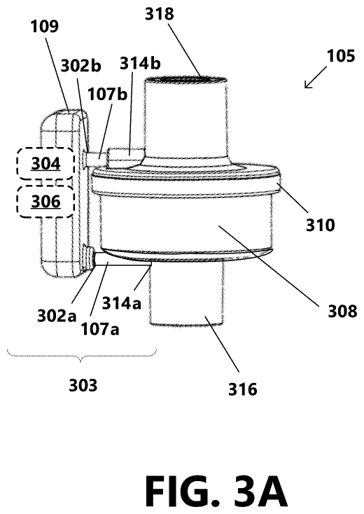

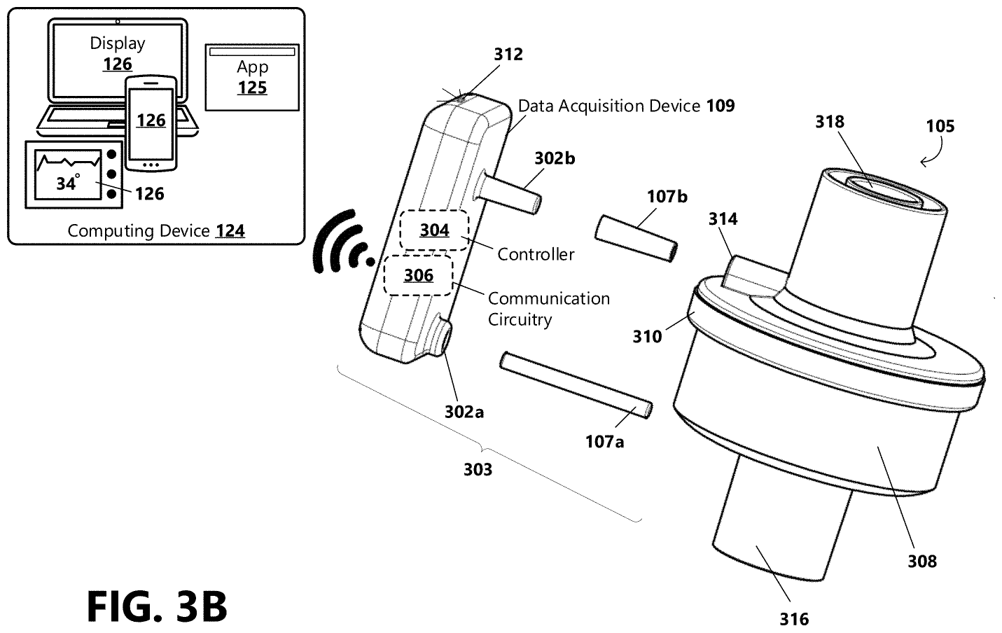

In some examples, the computing device 124 comprises a display 126 that includes a GUI via which the sensor measurements and/or derived useful information may be presented to a user. For example, the measurements and/or useful information may include characteristics of the breathing gases that may be presented in the form of graphs, wave representations, pie graphs, text, or other suitable forms of graphic display. Additionally or alternatively, sensor measurement data and/or useful information derived from the sensor measurement data may be further transmitted to a remote monitoring or parameter analytics system 136 communicatively coupled to the computing device 124 via one or a combination of wired or wireless networks 128. The filter 105, the data acquisition device 109, and the sensors 107 are described in further detail below with reference to FIGS. 2, 3A, and 3B.

In some examples, one or more sensors 107 may communicate with various components of the ventilation system 100, e.g., the pneumatic system 102, other sensors 107, the processor(s) 116, the controller 110, an optional humidification system 118, and/or any other suitable components and/or modules. A module as used herein refers to memory, one or more processors, storage, and/or other components of the type commonly found in command and control computing devices. In one aspect, the sensors 107 generate output and send this output to the data acquisition device 109, pneumatic system 102, other sensors 107, the processor 116, controller 110, humidification system 118, and/or any other suitable components and/or modules. The sensors 107 may employ any suitable sensory or derivative technique for obtaining one or more gas properties, patient parameters, and/or ventilator parameters associated with the ventilation of a patient 150.

The memory 112 includes non-transitory, computer-readable storage media that stores software that is executed by the processor 116 and which controls the operation of the ventilation system 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media can be implemented as any available non-transitory media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid-state memory technology, CD-ROM, DVD, or other optical storage, magnetic cassettes, magnetic tape, magnetic disk storage or other magnetic storage devices, or any other medium which can be used to store the desired information and which can be accessed by the computer.

FIG. 1 also shows the optional humidification system 118 as part of the ventilation system 100. For example, the humidification system 118 may operate to regulate humidity of the breathing gases. In some examples, the humidification system 118 can include a heating component to regulate temperature of the breathing gases. Additionally, although the humidification system 118 is shown interacting with specific components of the ventilation system 100 (e.g., the ventilation tubing system 130), the humidification system 118 may be located in other positions along the breathing circuit or ventilation tubing system 130 (e.g., along the inhalation limb 132, at the wye-fitting 170, or downstream of the wye-fitting 170).

In some examples, the ventilator of the ventilation system 100 may be considered to be the combination of the pneumatic system 102, the controller 110, and the display 122. For instance, the ventilator generates pressurized breathing gases that are delivered to the patient 150 via the ventilation tubing system 130.

As described above, various filters 105 and sensors 107 may be placed in various suitable locations. Some example filters 105 and sensors 107 and example locations where the filters 105 and sensors 107 may be incorporated in relation to other components of the ventilation system 100 are shown in FIG. 2. In the example shown in FIG. 2, a first filter 105a is shown attached to the ventilation system 100. For instance, the first filter 105a may be positioned between an example patient 150 and the wye-fitting 170. According to an example, a data acquisition device 109 may be attached to the first filter 105a, wherein the data acquisition device 109 may be paired, or in communication, with a computing device 124 for transmitting real time sensor measurement data wirelessly to an application 125 operating on the computing device 124. The data acquisition device 109 may be communicatively coupled to one or more of the sensors 107 integrated in or attached to the first filter 105a. In some examples, the one or more sensors 107 may be embedded into the first filter 105a during manufacture of the first filter 105a. In other examples, the one or more sensors 107 may be separate from and attached to the first filter 105a. That is, the one or more sensors 107 may be single-use elements that may be discarded with the first filter 105a after a single use; or, the one or more sensors 107 may be removeable from the first filter 105a and recycled for multi-use.

As illustrated, the first filter 105a may include one or more sensors 107a,b that may operate to obtain measurements related to characteristics of the exhaled breathing gases. In some examples, a flow rate of the exhaled gases may also be determined. For example, characteristics such as temperature, carbon dioxide level, humidity, and/or flow rate of the exhaled gases may be referred to as exhaled breathing gas characteristics. According to one example, the first filter 105a may include a first sensor 107a. The first sensor 107a, for example, may be a temperature sensor operative to measure the temperature of the patient's exhaled air. Further in the example shown, a second sensor 107b is also included in the first filter 105a. For instance, the second sensor 107b may be a carbon dioxide sensor operative to measure the carbon dioxide level of air exhaled from the patient 150. When the first sensor 107a and the second sensor 107b are in communication with the data acquisition device 109, temperature and carbon dioxide level measurements may be transmitted to the computing device 124. For instance, real time data (e.g., punctual data, average values, trends over time) may be read and transmitted to medical personnel to be analyzed. As should be appreciated, changes in heath parameters of the patient 150, such as the patient's body temperature (e.g., as can be determined by the patient's exhaled air temperature) and end-tidal CO₂ (ETCO2) (e.g., as can be determined by the level of carbon dioxide released at the end of expiration) may indicate changes in the patient's cardiac or pulmonary functions. Accordingly, by locating the first sensor 107a and the second sensor 107b in the first filter 105a that may be incorporated at a location near the patient 150 (e.g., between the patient 150 and the wye-fitting 170, the patient's temperature and/or carbon dioxide level measurements may be more accurate than temperature and/or carbon dioxide level measurements that may be taken farther away from the patient 150, and may provider a higher confidence level to medical personnel who may rely on such measurements to make decisions related to the care of the patient 150.

For instance, real-time gas properties that may be useful in treating the patient 150 may be obtained and transmitted wirelessly to medical personnel and/or a data analysis system (e.g., application 125, a remote monitoring or parameter analytics system 136) to be analyzed. As can be appreciated, ventilator use in emergency situations and/or in homecare, and/or utilization of low cost ventilation systems 100 that may not be equipped with such sensors 107a,b can be improved by incorporating a filter 105 in which the sensors 107a,b can obtain and provide gas properties and/or health parameter data for real-time data visualization and monitoring. Moreover, the ability to transmit real-time properties and/or parameters to a computing device 124 that is within short-range communication range of the data acquisition device 109 allows for a user of the computing device 124 to remotely monitor the patient 150, which may also increase safety by minimizing a risk of exposing medical caregivers to contagious diseases.

In some examples, one or more sensors 107 may be included in or attached to the inhalation limb 132, for example, to obtain measurements of delivered breathing gas characteristics. For instance, the inhalation limb 132 may include one or more filters 105 and/or one or more sets of sensors 107. In the example shown, the ventilation system 100 includes a second filter 105b located at a first end of the inhalation limb 132. The second filter 105b, for example, may be an inhalation module filter operative to filter breathing gases delivered to the patient 150. According to one example, the second filter 105b may be located external to the inhalation module 104 of the pneumatic system 102 as shown in FIG. 2, or according to another example, the second filter 105b may be embedded in the inhalation module 104, such as being adjacent to or incorporated into an inhalation port of the pneumatic system 102.

In some examples, the second filter 105b may include one or more sensors 107c-d. For instance, a third sensor 107c included in the second filter 105b may be a humidity sensor and a fourth sensor 107d included in the second filter 105b may be a temperature sensor. The third sensor 107c and the fourth sensor 107d may be configured to measure the humidity and temperature, respectively, of the delivered breathing air provided by the pneumatic system 102 of the ventilation system 100. According to one example, the second filter 105b may include a data acquisition device 109 in communication with the third sensor 107c and the fourth sensor 107b, and operative to transmit measurements obtained by the third sensor 107c and the fourth sensor 107b wirelessly to the computing device 124. According to another example, the third sensor 107c and the fourth sensor 107b may be in communication with a local datalogging system 204. The datalogging system 204, for example, may be configured to receive measurement data from one or more sensors 107 with which the datalogging system 204 may be communicatively connected. The received measurement data, in some examples, may be used in determining one or more ventilation system settings.

As illustrated in FIG. 2, in some examples, the ventilation system 100 may include a heating component, such as a heated tube 202, and a humidification system 118. For example, the humidity and the temperature of the breathing gas may be measured and controlled to prevent the patient's internal temperature from being at or below the dew point of the breathing gas. In other examples, there may be a therapeutic reason to set the target inhalation gas temperature above or below the internal temperature measurement. Other example ventilation systems 100 may not include a heating component or a humidification system 118. In some examples, a second humidity sensor (e.g., a fifth sensor 107e) and a second temperature sensor 107f (e.g., a sixth sensor 107f) may be located at a second end of the inhalation limb 132. For example, the fifth sensor 107e and the sixth sensor 107f may be configured to measure the humidity and temperature, respectively, of the delivered breathing air provided by the heating component and/or the humidification system 118. According to one example, the fifth sensor 107e and the sixth sensor 107f may be in communication with the local datalogging system 204. According to another example, the fifth sensor 107e and the sixth sensor 107f may be in communication with a data acquisition device 109 that may be integrated in the ventilation system 100 or attached to the ventilation system 100 and that may operate to wirelessly transmit measurements obtained by the fifth sensor 107e and the sixth sensor 107f to a computing device 124.

According to an example, another temperature sensor (e.g., a seventh sensor 107g), which may be associated with the humidification system 118, may be included in the ventilation system 100. For example, the seventh sensor 107g may operate to measure the resistance temperature of the humidifier heater. According to one example, the seventh sensor 107g may be in communication with the local datalogging system 204. According to another example, the seventh sensor 107g may be in communication with a data acquisition device 109 that may be integrated in the ventilation system 100 or attached to the ventilation system 100 and that may operate to wirelessly transmit temperature measurements obtained by the seventh sensor 107g to a computing device 124.

According to an example, another temperature sensor (e.g., an eighth sensor 107h), which may be associated with the heating system, may be included in the ventilation system 100. For example, the eighth sensor 107h may operate to measure the surface temperature of the heated tube 202 at a particular distance (e.g., 25 cm) from the patient 150. According to one example, the eighth sensor 107h may be in communication with the local datalogging system 204. According to another example, the eighth sensor 107h may be in communication with a data acquisition device 109 that may be integrated in the ventilation system 100 or attached to the ventilation system 100 and that may operate to wirelessly transmit temperature measurements obtained by the eighth sensor 107h to a computing device 124.

According to an example, another temperature sensor (e.g., a ninth sensor 107i) may be configured to measure the temperature of the ambient air in the environment in which the ventilation system 100 is operating. The ninth sensor 107i (e.g., the ambient air temperature sensor) may be positioned or coupled to any component in the ventilation system 100. In some examples, the ninth sensor 107i may be freestanding and not physically coupled to another component of the ventilation system 100. According to one example, the ninth sensor 107i may be in communication with the local datalogging system 204. According to another example, the ninth sensor 107i may be in communication with a data acquisition device 109 that may be integrated in the ventilation system 100 or attached to the ventilation system 100 and that may operate to wirelessly transmit temperature measurements obtained by the ninth sensor 107i to a computing device 124.

In some examples, one or more sensors 107 may be included in or attached to the exhalation limb 134. For instance, the exhalation limb 134 may include one or more filters 105 and/or one or more sets of sensors 107. In the example shown, the ventilation system 100 includes a third filter 105c located at a first end of the exhalation limb 134. The third filter 105c, for example, may be an exhalation module filter located at an exhalation port of the pneumatic system 102 and operative to filter breathing gases delivered to the patient 150. According to one example, the third filter 105c may be located external to the exhalation module 108 of the pneumatic system 102 as shown in FIG. 2, or according to another example, the third filter 105c may be embedded in the exhalation module 108. In some examples, the third filter 105c may include one or more sensors 107j-k. For instance, a tenth sensor 107j included in the third filter 105c may be a humidity sensor and an eleventh sensor 107k included in the third filter 105c may be a temperature sensor. The tenth sensor 107j and the eleventh sensor 107k may be configured to measure the humidity and temperature, respectively, at the end of the exhalation limb 134. According to one example, the third filter 105c may include a data acquisition device 109 in communication with the tenth sensor 107j and the eleventh sensor 107k, and operative to transmit measurements obtained by the tenth sensor 107j and the eleventh sensor 107k wirelessly to the computing device 124. According to another example, the tenth sensor 107j and the eleventh sensor 107k be in communication with the local datalogging system 204. As can be appreciated, in other examples, additional, fewer, or alternative sensors 107 and/or filters 105 may be included in the ventilation system 100.

FIG. 3A depicts a side view of an example filter 105, and FIG. 3B depicts an exploded view of the example filter 105. A sensor assembly 303 that may be coupled to or included with the filter 105. The sensor assembly 303 includes one or more sensors 107 and a data acquisition device 109 that interfaces with the one or more sensors 107 and communicates with an application 125 that may be configured to receive real-time sensor data transmitted by the data acquisition device 109. Various aspects of the filter 105, and the sensor assembly 303 including the sensor(s) 107 and data acquisition device 109 are described concurrently with reference to FIGS. 3A and 3B.

According to an example and as illustrated in FIGS. 3A and 3B, the filter 105 may be a heat and moisture exchanger (HME) comprising an antimicrobial filtration media or element 308 within the outer housing the filter 105. Other types of filtration elements 308 may also be utilized or implemented.

The filter 105 may be placed between the patient 150 and the wye-fitting 170 to filter breathing gases that are being delivered to the patient 150. The example filter 105 is configured to couple or attach to ventilation tubing on both sides or ends of the filter 105 such that gases flow through the filter 105. For example, the filter 105 includes a first end 316 and a second end 318. The first end 316 couples to one portion of the ventilation tubing or breathing circuit, and the second end 318 couples to another portion of the breathing circuit. Thus, gases may flow through the filter 105 in a direction from the first end 316 to the second end 318 or in a direction from the second end 318 to the first end 316. In some examples, the first end 316 may be referred to as an inlet, and the second end 318 may be referred to as an outlet, or vice versa. Where the filter 105 is positioned between the wye-fitting 170 and the patient interface 180, or incorporated into the patient interface 180, the filter 105 may filter the gas bidirectionally. For instance, when the patient 150 is inhaling, gas flows through the filter 105 in one direction, and when the patient 150 is exhaling, gas flows through the filter 105 in a second direction. In other examples, the filter 105 may be a unidirectional filter.

As the gases pass through the filter 105, the gases are filtered. For instance, the antimicrobial filtration media or element 308 may operate to filtrate airborne particles, thus reducing and preventing bacteria and microbes from propagating into the patient's airways. In some examples, the filter 105 may further include an electrostatic membrane 310 that may enhance electrostatic attraction of airborne particles to help improve filtration. In other examples, the filter 105 may be configured as and operate as a different type of filter for filtering the gases flowing through the ventilation tubing.

According to an aspect, the sensor assembly 303 includes at least one sensor 107 that may operate to detect or measure properties of gases flowing through the filter 105, such as temperature, carbon dioxide level, etc. In some examples, the sensor 107 is embedded into the filter 105, such as during manufacture of the filter 105 (e.g., during a molding phase of the filter 105). In such examples, the sensor 107 is incorporated into the housing of the filter, and an electrical contact may be exposed on the exterior of the housing to receive data from the sensor 107. In such examples, the filter 105 may be disposable and the embedded sensor 107 may be disposed of with the filter 105. According to other examples, the filter 105 may be manufactured to include at least one physical sensor interface that may operate to receive a sensor 107. For instance, the sensor 107 may be removable and configured to be inserted into and removed from the filter 105. In some examples, this may allow for sensors 107 and filters 105 to be interchangeable. That is, for instance, after use, one or more of the sensors 107 may be removed from the filter 105 and reused, and the filter 105 may be disposed.

In as the examples shown in FIGS. 3A and 3B, the filter 105 may include one or more ports 314a,b (generally, 314) configured to receive a sensor 107. The port(s) 314 may provide a sealed fit for the sensor(s) 107 such that the sensors 107 may be exposed to the breathing gases flowing through the filter 105, but preventing the breathing gases from escaping from the filter into the environment. In some examples, the ports 314 may include a through hole in the housing of the filter 105. The through hole is configured to receive at least a portion of the sensor 107. In the example depicted, a first sensor 107a and first port 314a are located upstream from the filtration media or element 308, and the second sensor 107b and the second port 314b are located downstream from the filtration media or element 308 (for examples where breathing gas flows from the first end 316 to the second end 318 of the filter 105). The sensor 107 may be press fit, screw fit, or otherwise inserted into the through hole of the port 314. Thus, the sensing elements of the sensors 107 may be exposed, via the through hole, to the gases flowing through the filter 105. In such examples, the sensors 107 may each have their own housing that houses or encloses the sensing elements, such as transducers that convert physical gas properties into an electrical signal. Accordingly, the sensor 107 includes a first portion that is exposed to the breathing gases and a second portion that exposes an electrical contact for communicating the output of the sensor 107.

The sensor assembly 303 also includes a data acquisition device 109 configured to collect various parameters/measurement data from sensors 107. The data acquisition device 109 is communicatively connected to the sensors 107, and the data acquisition device 109 transmits the measurement data wirelessly to a computing device 124. According to an example, the data acquisition device 109 may be a separate or removable component from the filter 105 and can be selectably attached to the filter 105. In one example, the data acquisition device 109 may be attached to the filter 105 via a press fit attachment, a screw attachment, or another attachment method. The data acquisition device 109 may comprise one or more physical input interfaces 302a,302b (generally, 302) that may be configured to connect to the one or more sensors 107 included in the filter 105.

The sensor assembly 303 illustrated in FIG. 3 is shown to include two (2) sensors 107a,107b and the data acquisition device 109 is shown to include two (2) corresponding physical input interfaces 302a,302b. In other examples, fewer or additional sensors 107 may be included in the filter 105 and more or fewer input interfaces 302a,302b may be included in the data acquisition device 109. According to one example, the one or more input interfaces 302 may operate to physically connect to the one or more sensors 107 for allowing an input of electrical signals to be received by the data acquisition device 109 from the one or more sensors 107 representative of the gas property measurements detected by the sensor(s) 107. For instance, one sensor 107a may be configured to sense the temperature of the patient's exhaled gases and produce electrical output signals or voltages that are a representation of the temperatures being sensed. The output signals or voltages may be received by the first input interface 302a. Additionally, another sensor 107b may be configured to sense the carbon dioxide level of the patient's exhaled gases and produce a discrete output signals or voltages that are a representation of the carbon dioxide levels being sensed, which may be received by the second input interface 302b. In some examples, the input interface 302 may be configured to activate the sensor 107 when the input interface 302 is connected to the sensor 107.

In the example depicted, each of the sensors 107 include an elongate housing having a length that is substantially greater than its width or diameter. A first end of the sensor 107 is inserted into (or received by) the respective port 314, and a second end of the sensor 107 is coupled to (or received by) the respective sensor input interface 302. Coupling the sensor 107 into the respective input interface 302 may form a physical and electrical connection between the data acquisition device 109 and the sensor 107. Thus, data may be communicated from the sensor 107 to the data acquisition device 109. In the example depicted, the sensors 107 are removable from both the data acquisition device 109 and the ports 314 of the housing of the filter 105. Thus, different types of sensors 107 may be interchanged and the sensors 107 may be reused and/or connected to different filters 105. Similarly, the data acquisition device 109 may be reused and/or different types of data acquisition devices may be utilized with sensors 107. In other examples, one or more of the sensors 107 may be permanently attached to the data acquisition device 109 and/or the body of the filter 105.

The data acquisition device 109 may include an outer housing which may enclose or provide a physical platform for additional electronics to process and/or communicate the data (e.g., electrical signals) received from the sensors. For instance, the outer housing includes the interfaces input interface 302 such that the sensors 107 may be connected to the housing, and the electronics of the data acquisition device 109 may be in electrical communication with the sensors 107.

For example, the data acquisition device 109 include a controller 304 that is in electrical communication with the one or more input interfaces 302. The controller 304 may read the output signals or voltages representative of the gas properties being sensed and facilitate the transmission of the sensor data to a paired computing device 124 via communication circuitry 306. The controller 304 and the communication circuitry 306 may be positioned or mounted within the housing of the data acquisition device 109.

The controller 304 may include one or more processors or processing circuits configured to process the data received from the sensors 107. The controller 304 may also include memory that stores instructions, that when executed by the processor(s) of the controller 304, cause the controller 304 to perform one or more of the operations described herein. In some examples, the controller 304 may further operate to convert the received discrete output signals or voltages into a different, or standardized, format prior to transmission. For instance, according to one example, the controller 304 may be configured to read analog voltage output from a sensor 107 and convert the voltage into a digital value, which may be transmitted to the computing device 124. In some examples, the data acquisition device 109 may further include a light or other indicator 312 on the exterior of the housing that may operate to indicate (e.g., flash or illuminate) when sensor data is transmitted to the computing device 124 and/or when the data acquisition device is in an “on” state. According to one example, the communication circuitry 306 may be embodied as a BLUETOOTH circuit or component that may operate to wirelessly pair with the computing device 124 and wirelessly transmit sensor data to the computing device 124 using BLUETOOTH or BLE protocols. In other examples, the communication circuitry 306 may communicate with the computing device 124 using ZIGBEE, ANT, Z-WAVE, or another close-proximity or short-range wireless communication protocol. In some examples, the data acquisition device 109 may communicate with the remote computing device 124 using longer or long-range communication protocols such as cellular data communications or wireless Internet protocols, such as WIFI protocols.

Accordingly, the sensor assembly 303 may include one or more sensors 107 that each have a housing the encloses sensing elements for converting a physical property of the breathing gases to an electronic signal. The housings of the sensors 107 are physically coupled to the housing of the filter 105 and a housing enclosing the controller 304 and the communication circuitry of the 306 of the data acquisition device 109. The housing of the filter 105 may be referred to as a filter housing and the housing the data acquisition device may be referred to as a data-acquisition housing.

As described previously, the computing device 124 may include a mobile computing device, a desktop computing device, a server computing device, a home-care ventilator device, a central monitoring station, a wearable computing device, or another type of computing device that may be configured to communicate wirelessly with the data acquisition device 109. In some examples, the computing device 124 may operate to execute an application 125 that may comprise BLUETOOTH or other wireless protocol communication capabilities to enable data communication between the application 125 and the data acquisition device 109. For instance, the application 125 may be configured to pair with the data acquisition device 109 and, once paired, receive sensor measurement data from the data acquisition device 109.

In some examples, the application 125 may be configured to display the received sensor data in a GUI on the display 126 of the computing device 124. In other examples, the application 125 may be configured to derive useful information based on the received sensor measurement data. In some examples, the computing device 124 may operate to pair with and receive sensor measurement data from a plurality of data acquisition devices 109. The plurality of data acquisition devices 109, for instance, may be associated with a single patient 150 or with a plurality of patients 150. According to an example, the application 125 or a remote monitoring or parameter analytics system 136 in communication with the application 125 may be configured to aggregate measurement data from multiple sensors 107 and/or from multiple filters 105, which may be linked to a single patient 150 or to a group of patients 150 (e.g., within a department of a healthcare facility, with a healthcare facility, across a group of healthcare facilities, within a region). Accordingly, real-time sensor measurements of gas properties that may be related to a patient's health during ventilation may be analyzed by medical personnel or a data analysis system (e.g., application 125) for making decisions related to the care of the patient 150.

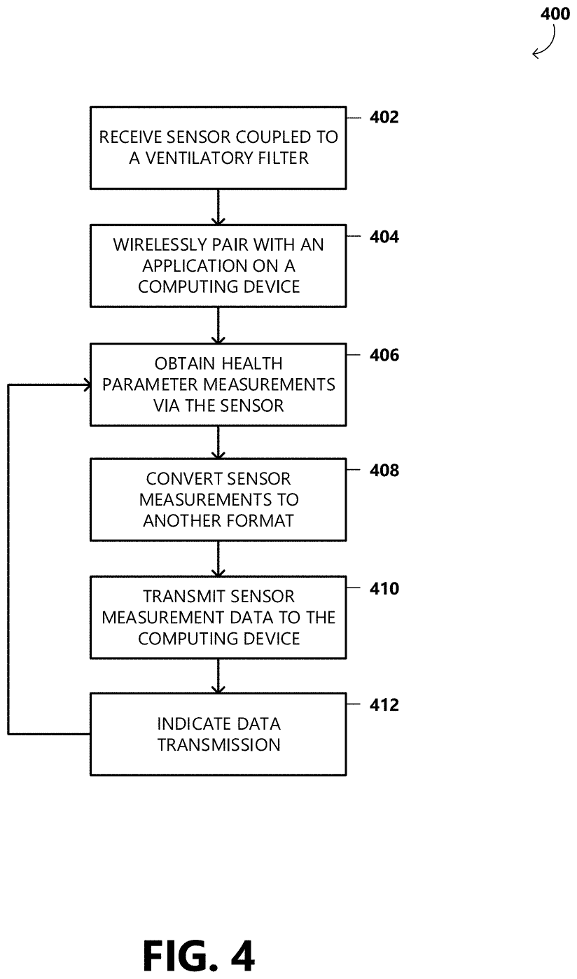

FIG. 4 shows an example method 400 according to the disclosed technology. The example method 400 includes operations that may be implemented or performed by the systems and devices disclosed herein. For example, the data acquisition device 109 depicted in FIGS. 1-3 may perform at least some of the operations described in the method 400. In addition, instructions for performing the operations of the methods disclosed herein may be stored in a memory that may be included in the controller 304 of the data acquisition device 109.

At OPERATION 402, a data acquisition device 109 may receive, or be connected to, at least one sensor 107 coupled to a ventilatory filter 105. The filter 105 may be attached or coupled to a component of a ventilation system 100. According to one example, the filter 105 may be attached between a patient 150 and the wye-fitting 170 of the ventilation system 100. In other examples, the filter 105 may be attached to the ventilation system 100 in another location. As described above, the data acquisition device 109 may include a controller 304 that may operate to receive discrete output signals or voltages output by the at least one sensor 107 via a connection with the at least one sensor 107 via at least one input interface 302.

At OPERATION 404, a wireless communication session may be established between the data acquisition device 109 and an application 125 executing on a computing device 124 wherein the computing device 124 may be within short-range wireless communication range of the data acquisition device 109. For example, the application 125 may be configured to establish a BLUETOOTH connection with the data acquisition device 109. As described above, the data acquisition device 109 may further include communication circuitry 306, such as a BLUETOOTH circuit, which may be used by the controller 304 of the data acquisition device to communicate with the application 125. For instance, the data acquisition device 109 may initiate or begin wireless communications with the computing device 124.

At OPERATION 406, the sensor 107 measures one or more gas properties and the discrete output signals or voltages representative of the gas property measurements being sensed by the at least one sensor 107 may be output by the at least one sensor 107 and received as input by the data acquisition device 109 via the at least one input interface 302. For instance, the data acquisition device 109 receives, from the sensor 107, the gas property data measured by the sensor 107. The gas property data may be received

At OPERATION 408, the gas property measurements received from the at least one sensor 107 may be converted into another format. For instance, if required or desired, the gas property measurements may be converted into digital values that are transmitted to the remote device 124 and/or application 125 at OPERATION 410. In some examples, an indication of the transmission of sensor data may be made at OPERATION 412. For instance, the data acquisition device 109 may include an indicator 312 that may be configured to illuminate when sensor data is transmitted.

In some examples, OPERATIONS 406-412 may repeat until the data acquisition device 109 is disconnected from the at least one sensor 107 or communicatively disconnected from the application 125. For example, real-time health parameter data may be provided to the application 125, which, in some examples, may operate to display the health parameter measurements in a GUI displayed on a display 126 associated with the computing device 124. In some examples, the application 125 may further operate to derive useful information based on the health parameter measurements and display the useful information in the GUI. In some examples, the health parameter measurements and/or derived useful information may be further transmitted to a remote monitoring or parameter analytics system 136 communicatively coupled to the computing device 124 via one or a combination of wired or wireless networks 128. According to an example, the measurements and/or useful information may include characteristics of the patient's exhaled breathing gases that may be presented in the form of graphs, wave representations, pie graphs, text, or other suitable forms of graphic display for real-time data visualization and monitoring.

Although the present disclosure discusses the implementation of these techniques in the context of a ventilator capable of providing ventilation support to a human patient, the techniques introduced above may be implemented for a variety of medical devices or devices utilizing filters 105. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A medical ventilatory filter comprising:
a first housing enclosing filtration media for filtering breathing gases flowing through the filter, the first housing defining a first port and a second port exposed to the breathing gases; and
a sensor assembly, the sensor assembly including:
a first sensor coupled to the first port, the first sensor configured to capture measurement data for a first gas property of breathing gases flowing through the filter;
a second sensor coupled to the second port, the second sensor configured to capture measurement data for a first gas property of the breathing gases flowing through the filter; and
a second housing including:
a processor, enclosed within the second housing, operative to process sensor data received from the first sensor and the second sensor;
communication circuitry, in communication with the processor, operative to wirelessly communicate the sensor data to a computing device located remotely from the filter;
a first physical input interface physically coupling the first sensor to the second housing and electrically coupling the first sensor and the processor; and
a second physical input interface physically coupling the second sensor to the second housing and electrically coupling the second sensor and the processor.

2. The filter of claim 1, wherein the first sensor is removable from at least one of the first housing or the second housing.

3. The filter of claim 1, wherein the first sensor is permanently embedded into the first housing.

4. The filter of claim 1, wherein the second housing is removably attached to at least one of the first sensor or the second sensor.

5. The filter of claim 1, wherein the first physical input interface allows for removing the first sensor from the second housing.

6. The filter of claim 1, wherein the first sensor is positioned upstream from the filtration media and the second sensor is positioned downstream from the filtration media.

7. The filter of claim 1, wherein the first sensor is one of a temperature sensor or a carbon dioxide sensor.

8. The filter of claim 7, wherein the first sensor is a carbon dioxide sensor.

9. The filter of claim 1, wherein a first end of the filter is configured to connect to first portion of a breathing circuit of a ventilation system and a second end of the filter is configured to connect to a second portion of the breathing circuit.

10. The filter of claim 1, wherein the filter is configured to connect to a breathing circuit of a ventilation system between a patient interface and a wye-fitting.

11. The filter of claim 1, wherein the first sensor is configured to measure gas properties associated with exhaled breathing gases.

12. A method for providing real-time gas property data of gases flowing through a medical ventilation system, the method comprising:
initiating, by a data acquisition device having a first housing enclosing a controller and wireless communication circuitry, wireless communication session with a remote application;
measuring, by a sensor physically coupled to the first housing and second housing of a filter enclosing filter media for filtering breathing gases flowing through the filter, a gas property of the breathing gases;
receiving, by the data acquisition device from the sensor, the measurement of the gas property of breathing gases; and
wirelessly transmitting, by the wireless communication circuitry, the received gas property measurement to the remote application.

13. The method of claim 12, wherein the sensor is removably connected to the first housing of the data acquisition device via an input interface of the first housing.

14. The method of claim 12, wherein receiving, from the sensor, the measurement of the gas property comprises receiving a temperature measurement of exhaled breathing gases.

15. The method of claim 14, wherein receiving, from the sensor, the measurement of the gas property comprises receiving a carbon dioxide level measurement of exhaled breathing gases.

16. The method of claim 14, wherein the filter is positioned between a patient interface and a wye-fitting of the ventilation system.

17. A ventilation system comprising:
a pneumatic system having an inhalation port and an exhalation port;
an inhalation limb connected to the inhalation port;
an exhalation limb connected to the exhalation port;
a wye-fitting connected to the inhalation limb and the exhalation limb;
a patient interface; and
a first filter positioned between a patient and the wye-fitting, the first filter comprising:
a first filter housing enclosing filtration media for filtering breathing gases flowing through the filter, the first filter housing defining a first port exposed to the breathing gases flowing through the first filter housing;
a first sensor coupled to the first port, the first sensor configured to capture measurement data for a first gas property of breathing gases flowing through the first filter; and
a first data-acquisition housing including:
a first processor, enclosed within the first data-acquisition housing, operative to process sensor data received from the first sensor;
first communication circuitry, in communication with the first processor, operative to wirelessly communicate the sensor data from the first sensor to a computing device located remotely from the ventilation system; and
a first physical input interface physically coupling the first sensor to the first data-acquisition housing and electrically coupling the first sensor and the first processor.

18. The ventilation system of claim 17, further comprising a second filter positioned on the exhalation limb between the wye-fitting and the pneumatic system, wherein the second filter comprises:
a second filter housing enclosing filtration media for filtering breathing gases flowing through the filter, the second filter housing defining a second port exposed to the breathing gases flowing through the second filter housing;
a second sensor coupled to the second port, the second sensor configured to capture measurement data for a second gas property of breathing gases flowing through the second filter; and
a first data-acquisition housing including:
a first processor, enclosed within the first data-acquisition housing, operative to process sensor data received from the first sensor;
first communication circuitry, in communication with the first processor, operative to wirelessly communicate the sensor data from the first sensor to a computing device located remotely from the ventilation system; and
a first physical input interface physically coupling the first sensor to the first data-acquisition housing and electrically coupling the first sensor and the first processor.

19. The ventilation system of claim 17, wherein the first sensor is removable from the first filter housing.

20. The ventilation system of claim 19, wherein the first data-acquisition housing is removable from the first sensor.
